(19) United States
(12) Patent Application Publication
(10) Pub. No.: US 2024/0247074 A1
(43) Pub. Date: Jul. 25, 2024

(54) THERAPEUTIC STRATEGY FOR NON-SMALL CELL LUNG CANCER AND OTHER CANCERS

(71) Applicant: New York University, New York, NY (US)

(72) Inventors: Jiehui Deng, Port Washington, NY (US); Kwok-Kin Wong, Arlington, MA (US); David Peng, Houston, TX (US); Benjamin Neel, New York, NY (US); Hsin-Yi Huang, New York, NY (US)

(21) Appl. No.: 18/417,928

(22) Filed: Jan. 19, 2024

Related U.S. Application Data
(60) Provisional application No. 63/439,987, filed on Jan. 19, 2023.

(51) Int. Cl. C07K 16/28; A61K 31/353; A61K 31/4245; A61K 31/454; A61K 31/4709; A61K 35/00; A61K 45/06; A61K 47/68

(52) U.S. Cl. CPC C07K 16/2896; A61K 31/353; A61K 31/4245; A61K 31/454; A61K 31/4709; A61K 35/00; A61K 45/06; A61K 47/6849

(57) ABSTRACT

Provided are methods for treatment of cancer by administering to an individual who has a cancer that includes cancer cells that have an LKBI mutation an anti-CD38 agent, an inhibitor of EP300, or a combination thereof. Methods are also provided for treating an individual who has a cancer that includes cancer cells that do not have an LKBI mutation a Salt-Inducible Kinase (SIK) inhibitor and an anti-CD38 agent. The SIK inhibitors sensitizes the cancer cells that do not have the LKBI mutation to the anti-CD38 agent.

### CROSS-REFERENCE TO RELATED APPLICATIONS

This application claims priority to U.S. provisional application No. 63/439,987, filed on Jan. 19, 2023, the entire disclosure of which is incorporated herein by reference.

### RELATED INFORMATION

The STK11 tumor suppressor gene encodes a serine-threonine kinase, LKB1, with pleiotropic functions in cell metabolism, polarity, and growth control. Germline loss-of-function STK11 mutations result in Peutz-Jeghers Syndrome (PJS), characterized by gastrointestinal polyposis and increased risk of various malignancies. Somatic mutations and deletions of LKB1 are observed in many sporadic cancers, including lung cancer. LKB1 defects are particularly important in lung adenocarcinoma, with ˜15-20% of cases exhibiting genomic inactivation of LKB1. This subset of non-small cell lung cancer (NSCLC) is associated with poor outcomes and reduced sensitivity to existing conventional chemotherapies and immunotherapies compared with LKB1 WT tumors. LKB1 phosphorylates and activates the 14 members of the AMPK-activated protein kinase (AMPK) subfamily, many of which are involved in metabolic regulation. Functional studies indicate that, among this group, the Salt-Inducible kinases (especially SIK1 and SIK3) are important for tumor suppression by LKB1, in keeping with the emerging importance of SIKs in growth regulation. Despite extensive preclinical and clinical studies of LKB1-mutant NSCLC, treatment of this subset of tumors remains a major unmet need in oncology. CD38 is a type II transmembrane receptor expressed in a variety of tissues, including lymphatic and myeloid cells, where it regulates cell proliferation and immune response, as well as in bronchial epithelial cells, pancreatic islet cells, and others. This multifunctional ectoenzyme serves as an NAD⁺ glycohydrolase and cyclic ADP-ribose synthase, and generates ADP-ribose—a metabolite also produced by other NAD⁺ consuming enzymes, including sirtuins (SIRT1-5) and poly(ADP-ribose) polymerases (PARPs). NAD⁺ is also an essential coenzyme that coordinates electron transfer critical for diverse metabolic redox reactions. CD38 has been shown to be a key regulator of global NAD⁺ availability through its ability to continuously degrade NAD⁺. CD38 is overexpressed in multiple myeloma, and anti-CD38 antibodies have substantial efficacy in the treatment of this malignancy in the clinic. Despite extensive studies of the biology of STK11-mutant NSCLC, there are currently no approved targeted therapies for this aggressive subtype. There is thus an ongoing and unmet need for approaches to targeting NSCLC cancers, and other cancers. The present disclosure is pertinent to this and other needs.

### BRIEF SUMMARY

The present disclosure reveals a novel therapeutic target CD38, that is highly expressed in LKB1 mutant lung cancer, and methods of targeting CD38. CD38 is a transmembrane glycoprotein that possesses a multi-functional enzyme activity that degrades NAD and modulates cellular NAD homeostasis. CD38 has pleiotropic functions in pathogenesis, affecting inflammation, obesity, diabetes, aging, and cancer. Anti-CD38 antibodies capable of antibody-dependent cytotoxicity (ADCC) have been approved by FDA for the treatment of multiple myeloma (MM), as myeloma cells highly and uniformly express CD38 on the cell surface. The disclosure demonstrates that LKB1 mutant cancers also show high cell surface expression of CD38. The disclosure demonstrates that SIK inhibitors sensitize LKB1 wild type tumors to anti-CD38 antibody treatment.

In non-limiting embodiments the disclosure provides methods for treatment of cancer, the method comprising: i) administering to an individual who has a cancer comprising cancer cells that have an LKBI mutation an anti-CD38 agent, an inhibitor of EP300, or a combination thereof; or ii) administering to an individual who has a cancer comprising cancer cells that do not have an LKBI mutation a Salt-Inducible Kinase (SIK) inhibitor and an anti-CD38 agent. The administration of the SIK inhibitor sensitizes the cancer cells that do not have the LKBI mutation to the anti-CD38 agent. In embodiments, performing i) or ii) inhibits growth of the cancer cells or activates an anti-cancer immune response, or produces a combination thereof. In embodiments, the individual is in need of treatment for lung cancer, colon cancer or pancreatic cancer. In an embodiment the lung cancer is non-small cell lung cancer (NSCLC).

In embodiments, the anti-CD38 agent is an anti-CD38 antibody that is any of Daratumumab, isatuximab, an anti-CD38 drug conjugate (CD38-ADC) such as STI-6129, or any other antibody constructs that target CD38, such as those known in the art as GBR 1342, TAK-079, TAK-169. As an alternative or in addition to antibody constructs, anti-CD38 drugs may be administered, non-limiting examples of which include and 78c and apigenin.

In embodiments, administering the inhibitor of EP300/CBP to the individual who has the cancer cells that have the LKBI mutation comprises administering any of CCS1477, A485, JQAD1, C646, SGC-CBP30/CBP30, CPI-637, EP31670/NEO2734, or I-CBP112.

In embodiments, when administering a combination of a SIK inhibitor and an anti-CD38 agent to the individual who has a cancer comprising cancer cells that do not have an LKBI mutation, the SIK inhibitor may be any of YKL-05-099, ARN-3236, MRT199665, YKL-06-061, YKL-06-062, GLPG3970, DB1113, RSS0680, Pterosin, MRIA9, or WH-4-023.

### DESCRIPTION OF THE FIGURES

The patent or application file contains at least one drawing executed in color. Copies of this patent or patent application publication with color drawing(s) will be provided by the Office upon request and payment of the necessary fee.

FIG. 1. LKB1 is a negative regulator of CD38 expression in lung cancer cells. (A, B), A series of KRAS mutant NSCLC cell lines with or without LKB1 mutation were analyzed for CD38 expression via fluorescence-activated cell sorting (FACS). (A), Representative FACS plots. (B), Quantification. (C), Western blot analysis of CD38 protein levels in KRAS mutant lung cancer cell lines with or without LKB1 mutation.

FIG. 2. LKB1-SIK signaling suppresses CD38 transcriptionally. (A), FACS analysis showing CD38 expression on KP lung tumors with sgRNA mediated targeting of Lkb1 or Sik family members versus control. (B), Representative FACS analysis of CD38 levels in KP lung cancer cells shown in panel A. (C), Western blot of CD38 levels in KP lines without Lkb1 or Sik family kinases expression, or KL lines without Lkb1 expression. (D), Relative CD38 mRNA levels in the lines shown in panel C.

FIG. 3. SIK inhibitor increases upregulation of CD38 in LKB1 wildtype tumors. Flow cytometric analysis of CD38 expression changes after SIK inhibitor YKL-05-099 treatment in lung cancer (KPC) (A), and colon cancer (CT26, MC38) or pancreatic cancer (KP1) lines (B) at the indicated time points and concentration.

FIG. 4. LKB1-SIK suppresses CD38 expression through CRE promoter (A), CREB reporter assay in the indicated cells. (B), Outline of transcriptional regulation of CD38 expression by LKB1/SIK signaling. Dephosphorylated CRTC activates CD38 transcription through CRE promoter after forming complex with CBP/P300 and CREB.

FIG. 5. Daratumumab induces killing of CD38 positive lung cancer. Mouse A549 (A-C) or HCC44 (D-F) xenograft tumor growth in response to daratumumab treatment. For each mouse, 3×10⁶ A549 cells (A) or 5×10⁵ HCC44 (D) were implanted into nu/nu mice subcutaneously. Daratumumab or hIgG1 (12.5 mg/kg, once per week) was dosed when tumor size reached 100 mm³. Growth curve of A549 cells (A) or HCC44 (D) after daratumumab treatment. B-C, Individual mouse tumor growth curve for A549 xenografts treated with either hIgG1 (B) or Daratumumab (C) shown in panel A. (E-F), Individual mouse tumor growth curve for HCC44 xenograft treated with either hIgG1 (E) or Daratumumab (F) shown in panel D.

FIG. 6. EP300 inhibition suppresses LKB1 mutant tumor progression. (A), EP300 inhibitors CCS1477 and A485 suppresses CD38 expression in LKB1 mutant tumors. (B), Representative FACS analysis of CD38 expression after drug treatment. (C), Tumor volume changes of KL tumors after indicated EP300 inhibitors treatment. (D). Tumor growth curve of KL tumors treated with CCS1477 or A485.

### DESCRIPTION

Unless defined otherwise, all technical and scientific terms used herein have the same meaning as commonly understood by one of ordinary skill in the art to which this invention belongs.

Throughout this application, the use of the singular form encompasses the plural form and vice versa. For example, “a”, or “an” also includes a plurality of the referenced items, unless otherwise indicated.

Where a range of values is provided in this disclosure, it should be understood that each intervening value, and all intervening ranges, between the upper and lower limit of that range is also included, unless clearly indicated otherwise. The upper and lower limits from within the broad range may independently be included in the smaller ranges encompassed within the disclosure.

The disclosure includes all polynucleotides and amino acid sequences described herein.

The present disclosure provides methods for treatment of cancer. In embodiments the methods comprise: i) administering to an individual who has a cancer comprising cancer cells that have an LKBI mutation an anti-CD38 antibody, an inhibitor of EP300, or a combination thereof; or ii) administering to an individual who has a cancer comprising cancer cells that do not have an LKBI mutation one or more Salt-Inducible Kinase (SIK) inhibitors and an anti-CD38 antibody, wherein administration of the one or more SIK inhibitors sensitizes the cancer cells that do not have the LKBI mutation to the anti-CD38 antibody. Implementing i) or ii) inhibits growth of the cancer cells or activates an immune response against cancer, or both. Activating an immune response includes but is not necessarily limited to promoting a cell-mediated anti-cancer immune response in the individual.

The disclosure includes the proviso that any described method may exclude administration of an immune based therapy to the individual. Thus, any described method may exclude administration of an immune based therapy that comprises, for example, an immune checkpoint inhibitor, including but not necessarily limited to checkpoint inhibitors directed against PD-1, PD-L1, LAG-3, Tim-1, 41BB, Ox40 and CD122. The disclosure also includes the proviso that any described method may exclude administration of any one or a combination of inhibitors of NAMPT, HDAC4, SHP2 and CXCR1/2. Accordingly, in embodiments, a method of this disclosure may consist of the described steps and described agents and/or combinations of agents that target CD38, EP300, or a SIK inhibitor. As a non-limiting example, when administering an anti-CD38 agent to an individual who has a cancer comprising cancer cells that have an LKBI mutation, the anti-CD38 agent may be the only therapeutically effective agent that is administered, or it may be administered in combination with an inhibitor of EP300/CBP, or other agents that are not expressly excluded as discussed above.

In embodiments an effective amount of an agent described herein is administered to an individual in need thereof. In embodiments, an effective amount is an amount that reduces one or more signs or symptoms of a disease and/or reduces the severity of the disease. An effective amount may also inhibit or prevent the onset of a disease or a disease relapse. A precise dosage can be selected by the individual physician in view of the patient to be treated. Dosage and administration can be adjusted to provide sufficient levels of binding partner to maintain the desired effect. Additional factors that may be considered include the severity and type of the disease state, age, weight, and gender of the patient, desired duration of treatment, method of administration, time and frequency of administration, drug combination(s), reaction sensitivities, and/or tolerance/response to therapy. In embodiments implementing a described method inhibits cancer cell growth, inhibits establishment of a primary tumor, inhibitors tumor growth, inhibits metastasis, or a combination thereof.

The described methods may include testing a biological sample from the individual to determine the presence, type, or absence of an LKBI mutation. In a non-limiting approach, the method comprises selecting an individual who has a cancer comprising cancer cells that have an LKBI mutation. The selected individual is treated with an anti-CD38 antibody, an inhibitor of EP300, or a combination thereof. In another non-limiting approach, the method comprises selecting an individual who has a cancer comprising cancer cells that do not have an LKBI mutation and treating the individual with one or more Salt-Inducible Kinase (SIK) inhibitors and an anti-CD38 antibody. Administration of the one or more SIK inhibitors sensitizes the cancer cells that do not have the LKBI mutation to the anti-CD38 antibody.

In embodiments, the anti-CD38 agent and another agent as described herein may be administered at the same or different times, over the same period of time or different periods of time, by the same route or different routes, concurrently or sequentially, or overlapping, or any combination of administration regimens and routes. In one embodiment, a SIK inhibitor is administered first to sensitize LKBI wild type tumors, and subsequently an anti-CD38 agent is administered.

The described anti-cancer agents may be used as pharmaceutical formulations. The pharmaceutical compositions may be in the form of solutions, suspensions, emulsions, and solid injectable compositions that are dissolved or suspended in a solvent immediately before use. The injections may be prepared by dissolving, suspending, or emulsifying one or more of the active ingredients in a diluent. Examples of diluents are distilled water for injection, physiological saline, physiologic buffer, vegetable oil, alcohol, and a combination thereof. Further, the compositions may contain stabilizers, solubilizers, suspending agents, emulsifiers, soothing agents, buffers, preservatives, etc. The pharmaceutical compositions may be formulated into a sterile solid or powdered preparation, for example, by freeze-drying, and may be used after sterilized or dissolved in sterile injectable water or other sterile diluent(s) immediately before use. The compositions can include one or more standard pharmaceutically acceptable carriers. Some examples herein of pharmaceutically acceptable carriers can be found in: Remington: The Science and Practice of Pharmacy (2013) 22nd Edition, Pharmaceutical Press.

The pharmaceutical compositions of the invention may be administered via any route that is appropriate, including but not limited to oral, parenteral, sublingual, transdermal, rectal, transmucosal, topical, via inhalation, via buccal administration, or combinations thereof. Parenteral administration includes, but is not limited to, intravenous, intraarterial, intraperitoneal, subcutaneous, intratumoral, intramuscular, intrathecal, and intraarticular. The agents(s) can also be administered in the form of an implant, which allows a slow release of the compound(s), as well as a slow controlled i.v. infusion.

In embodiments the cancer the individual who is treated according to the described methods any type of tumor or blood cancers. If the cancer is NSCLC, it may be any of squamous cell (or epidermoid) carcinoma, adenocarcinoma and, large cell (or undifferentiated) carcinoma. In embodiments, the cancer is melanoma, kidney cancer, bladder cancer, liver cancer, pancreatic cancer, colon cancer, head and neck cancers, prostate cancer, ovarian cancer, cervical cancer, Hodgkin lymphoma, urinary tract cancers, and other types of cancers. The cancer may be refractory to previously available treatments.

With respect to LKB1, this gene not only encodes a serine/threonine kinase that reprograms the cellular metabolism through AMPK-related kinases, especially SIK family kinases, but also plays a pleiotropic role in tumor growth, metastasis, and immune evasion. The disclosure reveals a mechanistic link between loss of function in LKB1-SIK signaling and transcriptional upregulation of CD38. The resulting increase of CD38 expression at the cell correlated with an elevation in the product of NAD+ metabolism, ADP-ribose. In vivo treatment of LKB1 mutant lung tumors with a CD38 targeting monoclonal antibody arrested tumor growth. Thus, CD38 represents a target unique to LKB1 mutant cancers including but not limited to NSCLC. The disclosure accordingly provides approaches to targeting CD38 through EP300 inhibitors, or sensitizing LKB1 wildtype tumors for anti-CD38 therapy.

The nucleotide sequence of the human LKB1 gene (also referred to as STK11) is known in the art, such as from Gene ID: 679, from which the nucleotide sequences and amino acid sequences are incorporated herein by reference as they exist in the database as of the effective filing date of this application. LKB1 mutations (or the absence thereof) in cancer cells can be identified by those skilled in the art using routine approaches.

In one embodiment all of the cancer cells that are treated using a described method have an LKB1 mutation. In another embodiment, all of the cancer cells that are treated using a described method lack an LKB1 mutation. In an embodiment an LKB1 mutation as referred to herein comprises a loss-of-function mutation, such mutations being recognized by those skilled in the art.

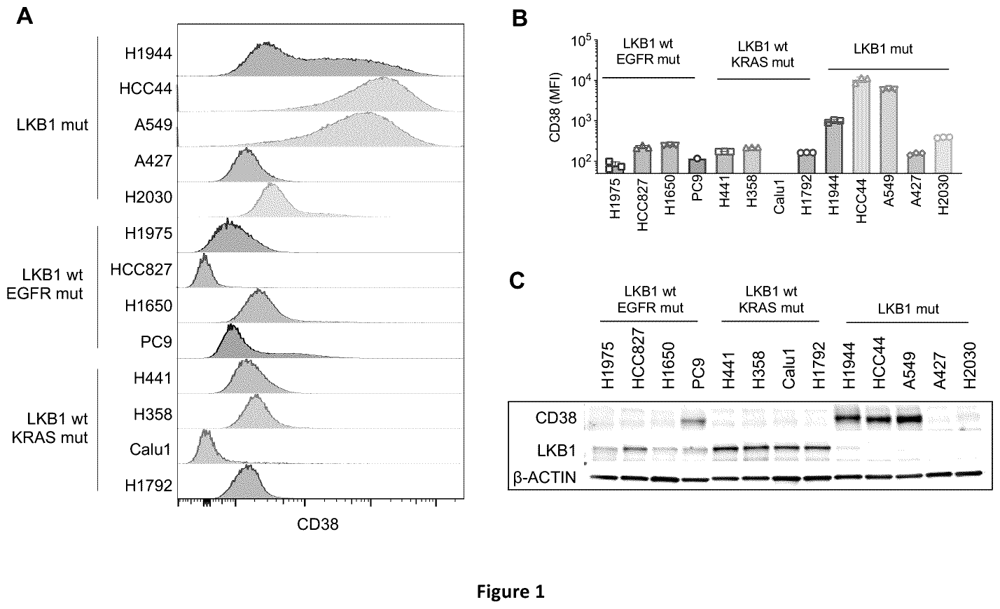

In this disclosure we used a series of human KRAS mutant NSCLC cell lines with or without LKB1 alterations to analyze the relationship between LKB1 mutations and CD38 expression. CD38 surface levels were markedly (5˜50-fold) higher in 3 of 4 lines with genomic inactivation of LKB1-HCC44 (frameshift insertion), A549 (nonsense mutation), and H1944 (missense mutation, K62N) compared with LKB1 WT lines (H441, H358, Calu1, and H1792) (FIG. 1). Restoring WT LKB1 expression to A549 cells suppressed CD38 expression, whereas a kinase-dead mutant of LKB1 (LKB1-KD) failed to do so. Thus, and without intending to be bound by any particular theory, it is considered that LKB1 is a negative regulator of CD38, as shown in FIG. 1.

The disclosure includes analysis of the mechanism by which LKB1 regulates CD38, focusing on the SIK family kinases given their role in LKB1-mediated tumor suppression. LKB1 activates SIKs by phosphorylating a threonine residue in the T-loop of their kinase domains. Accordingly, global phosphoproteomic analysis of human NSCLCs showed that LKB1 mutant tumors have significantly lower levels of activating SIK phosphorylation (gauged by SIK3-T221 phosphorylation) than other LKB1 wildtype lung tumors in the Clinical Proteomic Tumor Analysis Consortium (CPTAC) dataset. Consistent with decreased SIK kinase activity and the feedback loop between SIK inactivation and transcriptional upregulation of SIK genes, we found that SIK1 and SIK2 mRNA levels were increased in human KL tumors compared with KP tumors. Expression of the entire set of 14 LKB1 downstream AMPK/SIK kinases was shown for reference and demonstrated that select other family members are also differentially expressed (e.g., Ampk/Prkaa1). Paralleling the human data, Sik1 and Sik2 were elevated in KL murine lung tumors and cell lines, along with Sik3 and Ampk/Prkaa1.

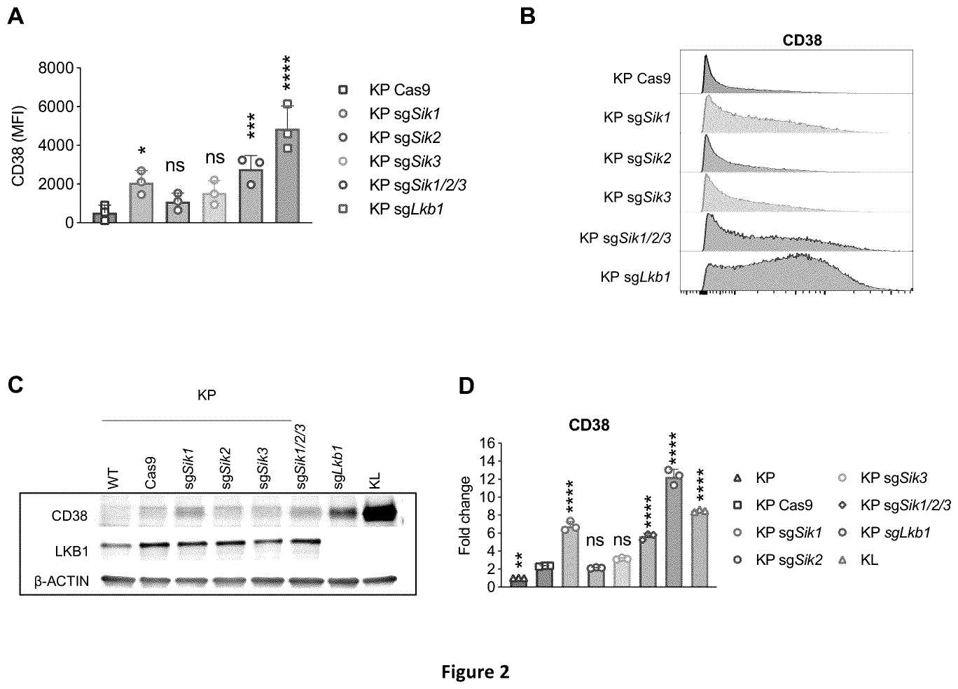

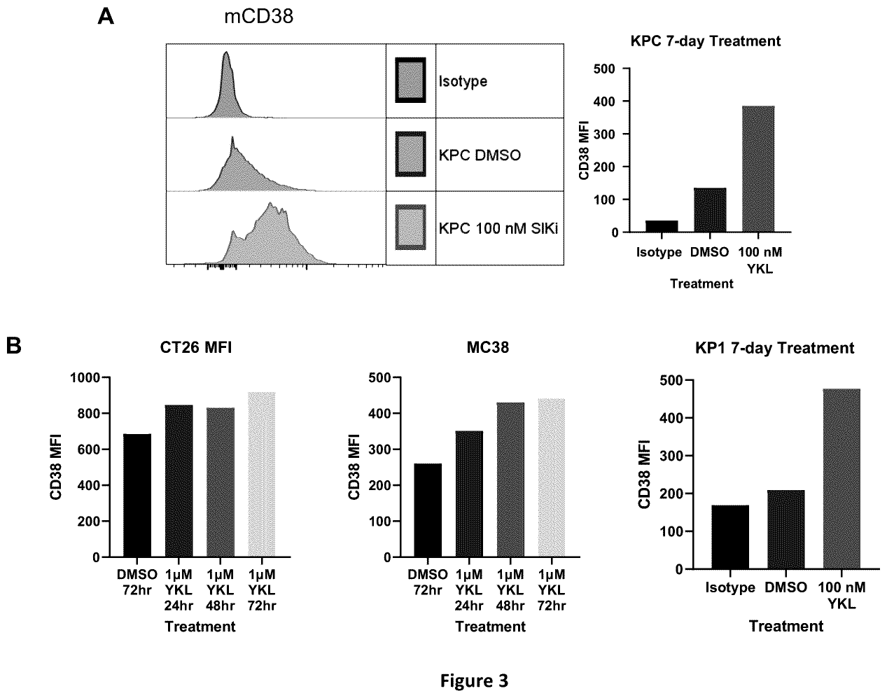

In line with the results of the LKB1 rescue studies, CRISPR-mediated knockout of Lkb1 in murine KP NSCLC cells led to increased cell surface expression of CD38 (FIG. 2A, 2B). We subsequently tested the impact of knockout of Sik1, Sik2 and Sik3, alone or in combination. We observed prominent increases in CD38 expression at the cell surface in KP cells upon inactivation of Sik1 or of all three SIK family members, but not to the level of CD38 in KP sgLkb1 cells (FIG. 2A, 2B). Without intending to be constrained by any particular theory, it is considered this indicates other factors other than SIK1/2/3 downstream of LKB1, might also contribute to the suppression of CD38 expression. LKB1 loss also induced Sik1 mRNA expression, consistent with the aforementioned feedback circuit for Sik1 transcriptional regulation. LKB1 or SIK loss also upregulated total CD38 protein levels and induced CD38 mRNA expression as determined by western blot and real-time PCR analysis, respectively (FIG. 2C, 2D). Likewise, acute pharmacologic inhibition of SIK kinase activity with the pan-SIK inhibitor, YKL-05-099, upregulated CD38 in multiple LKB1 WT mouse cancer cell lines, including lung cancer, colon cancer and pancreatic cancer cell lines (FIG. 3). These data indicate that LKB1 suppresses CD38 expression level through SIK family kinases, mainly through SIK1.

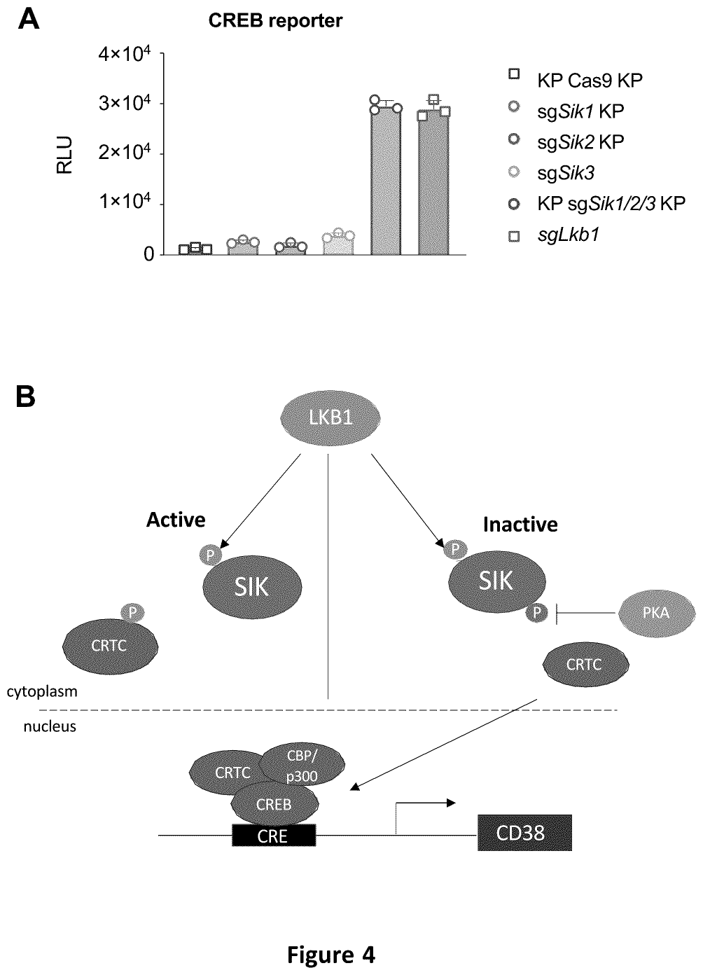

The CREB Regulated Transcription Coactivator family (CRTC1, CRTC2, and CRTC3) proteins are known targets of SIK. SIK-mediated phosphorylation of CRTCs at a 14-3-3 motif leads to their cytoplasmic retention, whereas loss of this phosphorylation due to LKB1 or SIK inactivation causes CRTC nuclear translocation and complex formation with cAMP response element binding protein (CREB) and CREB binding protein (CBP/p300) to initiate gene transcription through cAMP response element (CRE) binding sites. Analysis of the ENCODE ChIP-seq database revealed binding of p300 (a.k.a. EP300) to the CD38 promoter at the CRE site. Moreover, analysis of the CREB transcriptional activity using a reporter with a synthetic CRE motif demonstrated that LKB1 or SIK1/2/3 loss in murine KP tumor cells resulted in markedly increased CREB reporter activity (FIG. 4A). These results are consistent with CRTC/CREB/P300 activation mediating CD38 induction upon LKB1 or SIK loss of function (FIG. 4B). Collectively, the data indicate that inactivation of LKB1/SIK signaling drives CREB-mediated transcriptional upregulation of the NAD-consuming enzyme CD38 in vivo.

The disclosure includes analysis of whether the increase in CD38 in LKB1 mutant cancers confer an emergent vulnerability that could be exploited therapeutically. Multiple drugs targeting CD38, including the monoclonal antibodies daratumumab and isatuximab, are approved for use in multiple myeloma (MM), a plasma cell malignancy characterized by high CD38 expression on the tumor cell surface. Daratumumab is the first approved antibody against CD38, which is a fully human IgG1-κ monoclonal antibody that kills MM cells through multiple mechanisms, including antibody-dependent cellular cytotoxicity (ADCC) via binding to Fcγ receptors (FcγR).

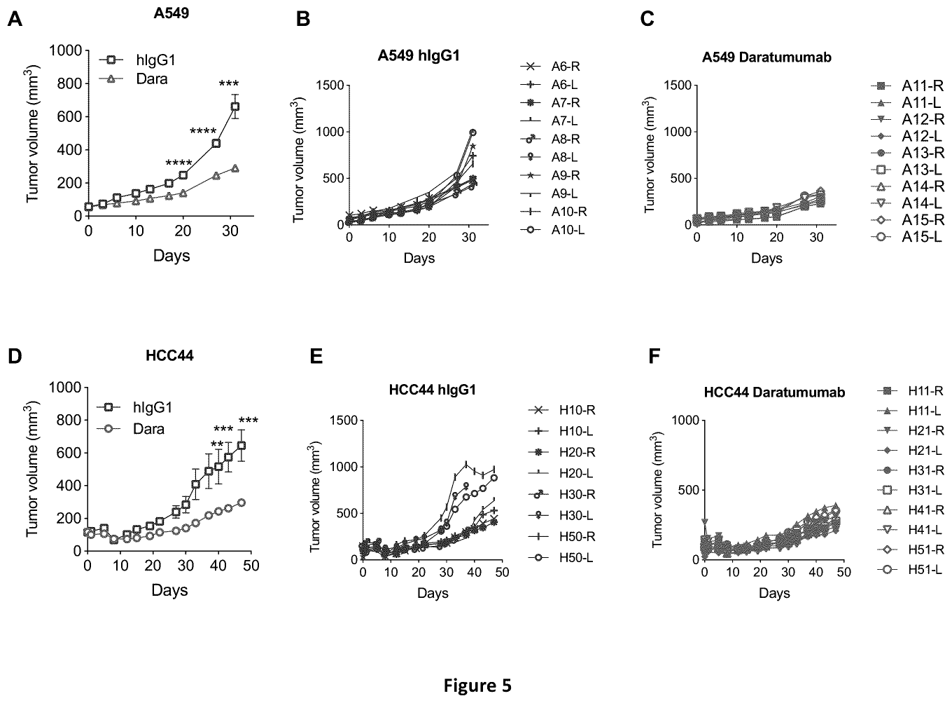

To study the effects of daratumumab in vivo, we implanted the LKB1 mutant A549 and HCC44 cell lines into athymic nude (nu/nu) mice to generate tumor xenografts. Daratumumab is a human IgG1, which can be recognized by mouse FcγRs on effector cells, resulting in ADCC induced between daratumumab with mouse NK cells through crosstalk, enabling evaluation of NK cell-mediated tumor cell killing in vivo. When tumors reached approximately 100 mm³ in volume, mice were treated with daratumumab once weekly. This led to a significant reduction in tumor growth in both mutant LKB1 tumor models (FIG. 5), supporting use of CD38 as a target in these tumors.

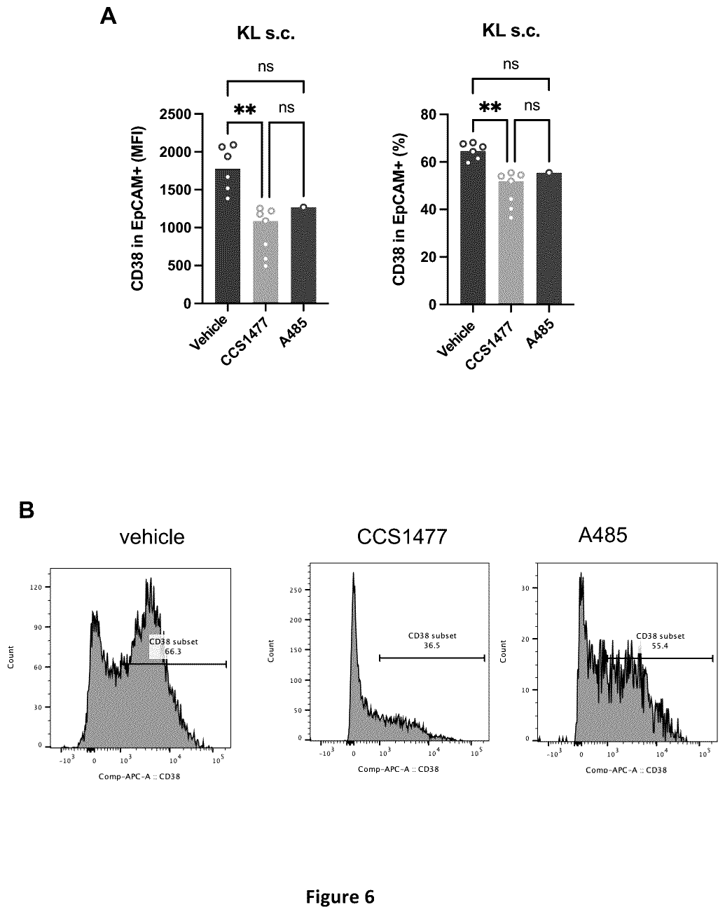

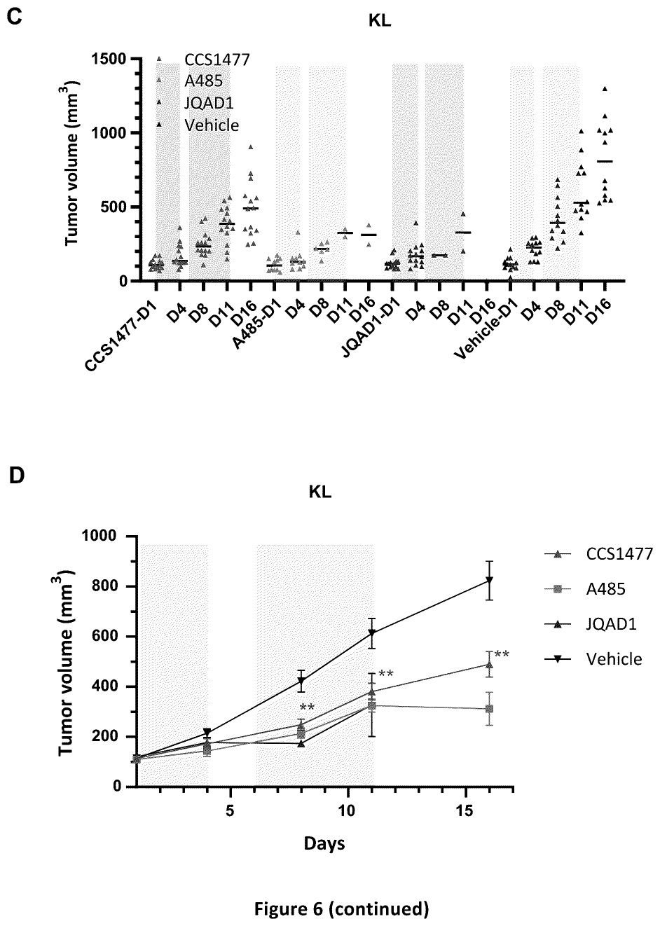

Since LKB1/SIK suppresses CD38 expression through CTRC/EP300/CREB complex, we further tested inhibition of CD38 via EP300 inhibitors, including CCS1477 and A485, in LKB1 mutant lung cancer tumors. When EP300 is inhibited, there is a decrease of CD38 expression on the tumor cells (FIG. 6A, 6B), and a reduced tumor growth in vivo (FIG. 6C, 6D).

Despite extensive studies of the biology of STK11-mutant NSCLC, there are no previously available targeted therapies for this aggressive subtype. LKB1 not only encodes a serine/threonine kinase that reprograms the cellular metabolism through AMPK-related kinases, especially SIK family kinases, but also plays a pleiotropic role in tumor growth, metastasis, and immune evasion. The disclosure reveals a mechanistic link between loss of function in LKB1-SIK signaling and transcriptional upregulation of CD38. The resulting increase of CD38 expression at the cell correlated with an elevation in the product of NAD+ metabolism, ADP-ribose. In vivo treatment of LKB1 mutant lung tumors with a CD38 targeting monoclonal antibody arrested tumor growth.

Other embodiments of the disclosure will be apparent to those skilled in the art from consideration of the specification and practice of the disclosure disclosed herein. It is intended that the specification and examples be considered as exemplary only.

What is claimed is:

1. A method for treatment of cancer, the method comprising:
i) administering to an individual who has a cancer comprising cancer cells that have an LKBI mutation an anti-CD38 agent, an inhibitor of EP300/CBP, or a combination thereof; or
ii) administering to an individual who has a cancer comprising cancer cells that do not have an LKBI mutation a Salt-Inducible Kinase (SIK) inhibitor and an anti-CD38 agent, wherein administration of the SIK inhibitor sensitizes the cancer cells that do not have the LKBI mutation to the anti-CD38 agent; and
wherein i) or ii) inhibits growth of the cancer cells and/or activates an anti-cancer immune response.

2. The method of claim 1, wherein the cancer is lung cancer.

3. The method of claim 1, wherein the cancer is non-small cell lung cancer (NSCLC).

4. The method of claim 1, comprising administering the anti-CD38 agent, wherein the anti-CD38 agent is an anti-CD38 antibody or CD38 inhibitor to the individual who has the cancer cells that have the LKBI mutation, and wherein the anti-CD38 agent is selected from the group consisting of daratumumab, isatuximab, an anti CD38-antibody drug conjugate, GBR 1342, TAK-079, TAK-169, 78c, apigenin, or a combination thereof.

5. The method of claim 1, comprising administering the inhibitor of EP300/CBP to the individual who has the cancer cells that have the LKBI mutation, and wherein the EP300/CBP inhibitor is selected from the group consisting of CCS1477, A485, JQAD1, C646, SGC-CBP30/CBP30, CPI-637, EP31670/NEO2734, I-CBP112, or a combination thereof.

6. The method of claim 1, comprising administering a combination of the anti-CD38 agent and the inhibitor of EP300/CBP to the individual who has the cancer cells that have the LKBI mutation.

7. The method of claim 1, comprising administering a combination of a SIK inhibitor and an anti-CD38 agent to the individual who has a cancer comprising cancer cells that do not have an LKBI mutation, and wherein the SIK inhibitor is selected from the group consisting of YKL-05-099, ARN-3236, MRT199665, YKL-06-061, YKL-06-062, GLPG3970, DB1113, RSS0680, Pterosin, MRIA9, WH-4-023, or a combination thereof.

8. A method comprising selecting an individual who has a cancer comprising cancer cells that have an LKBI mutation, and treating the individual with an anti-CD38 agent, an inhibitor of EP300/CBP, or a combination thereof.

9. The method of claim 8, comprising treating the individual with the anti-CD38 agent alone.

10. The method of claim 9, wherein the anti-CD38 agent is selected from the group consisting of daratumumab, isatuximab, an anti CD38-antibody drug conjugate, GBR 1342, TAK-079, TAK-169, 78c, apigenin, or a combination thereof.

11. The method of claim 8, comprising treating the individual with the inhibitor of EP300/CBP.

12. The method of claim 11, wherein the inhibitor of the EP300/CBP is selected from the group consisting of CCS1477, A485, JQAD1, C646, SGC-CBP30/CBP30, CPI-637, EP31670/NEO2734, I-CBP112, or a combination thereof.

13. The method of claim 8, comprising administering to the individual a combination of the anti-CD38 agent and the inhibitor of EP300/CBP.

14. A method comprising selecting an individual who has a cancer comprising cancer cells that do not have an LKBI mutation and administering to the individual a Salt-Inducible Kinase (SIK) inhibitor and an anti-CD38 agent to thereby treat the cancer.

15. The method of claim 14, wherein administration of a SIK inhibitor sensitizes the cancer cells that do not have the LKBI mutation to the anti-CD38 agent.

16. The method of claim 14, wherein the SIK inhibitor is selected from the group consisting of YKL-05-099, ARN-3236, MRT199665, YKL-06-061, YKL-06-062, GLPG3970, DB1113, RSS0680, Pterosin, MRIA9, WH-4-023, or a combination thereof.

17. The method of claim 14, wherein the anti-CD38 agent is selected from the group consisting of daratumumab, isatuximab, an anti CD38-antibody drug conjugate, GBR 1342, TAK-079, TAK-169, 78c, apigenin, or a combination thereof.
